(19) United States
(12) Patent Application Publication
(10) Pub. No.: US 2004/0087874 A1
(43) Pub. Date: May 6, 2004

(54) Saliva collection system

(76) Inventor: David Schneider, Royal Oak, MI (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/695,329

(22) Filed: Oct. 28, 2003

Related U.S. Application Data
(60) Provisional application No. 60/421,823, filed on Oct. 28, 2002.

(51) Int. Cl. A61B 5/00

(52) U.S. Cl. 600/573

(57) ABSTRACT

A kit and method for collecting a saliva sample for subsequent biologic assay that avoids collection contact with the biohazardous saliva sample. The kit includes a container, a preservative solution that is retained in the container and a saliva collection device. A subsequent laboratory assay performed by conventional techniques specific to the biologic of interest provides results comparable to those obtained from blood or urine samples. The kit allows an employee to remain at his or her place of business and requires only a minor inconvenience during testing.

### REFERENCE TO RELATED APPLICATIONS

The application claims priority from U.S. Provisional Patent Application No. 60/421,823, filed Oct. 28, 2002.

### BACKGROUND OF THE INVENTION

1. Field of the Invention

The present invention is directed to a system for the collection of saliva. More particularly, the invention is directed to a system for the collection of a saliva sample for use in diagnostic testing and the preservation of that sample.

2. Reference to Related Art

Drug and alcohol addiction is a widespread problem that can destroy the addicted individual and adversely affecting those close to them. Employers too are susceptible to the deleterious effects of addiction. In the modern workplace, focused and efficient employees are essential for employers that wish to maintain high quality, productivity and safety while minimizing costs and absenteeism. In order for employees to attain and sustain such high standards, it is crucial that each employee be both healthy and alert. An employee who is in poor health or that is inattentive reduces efficiency and may increase the risk of injury to themselves and other employees.

In an effort to combat drug and alcohol abuse in the workplace, many employers require employees to undergo mandatory drug testing. These tests generally require the employee to leave his or her place of business and travel to a test facility. Alternatively, the testing might take place at the workplace. However, since many of these tests require a urine sample, it is generally always necessary to provide the tested employee with at least a minimum level of privacy regardless of the location where the test is administered. Therefore, the present manner of testing results in at least two problems. First, the employee is required to leave his or her job to undergo testing when they could otherwise be working. Second, the privacy required by urine tests affords the employee an opportunity to submit a fraudulent sample (e.g., the employee could obtain a sample from another person and submit that drug-free sample for testing).

It would be desirable for an employer to have a system for testing an employee at its workplace. The test should require only a minimum level of personal inconvenience to the employee. It would also be advantageous if such a system would preserve the employee's sample for later forensic testing (e.g., to determine an individual's blood alcohol or other drug level).

The instant invention provides a kit and method for collecting a saliva sample for subsequent biologic assay that avoids collection contact with the biohazardous saliva sample. A subsequent laboratory assay performed by conventional techniques specific to the biologic of interest provides results comparable to those obtained from blood or urine samples. The kit allows an employee to remain at his or her place of business and requires only a minor inconvenience during testing. The system also provides the necessary safeguards against submission of fraudulent tests while insuring that an accurate and precise drug test can be taken later.

### SUMMARY OF THE INVENTION

A kit and method for collecting a saliva sample for subsequent biologic assay that avoids collection contact with the saliva sample. The kit includes a container, a preservative solution that is retained in the container and a saliva collection device. The container may include a resealable, transfer vial. The saliva collection device may include a transfer pipette, the end of which includes a flavored salivation catalyst. Once collected, a subsequent laboratory assay performed by conventional techniques specific to the biologic of interest provides results comparable to those obtained from blood or urine samples. The kit allows an employee to remain at his or her place of business and requires only a minor inconvenience during testing.

### BRIEF DESCRIPTION OF THE DRAWINGS

Reference will now be had to the attached drawings wherein like reference numerals refer to like parts throughout and wherein:

FIG. 1, is an environmental perspective view of a saliva collection system constructed in accordance with the present invention during use;

FIG. 2, is an perspective view of a transfer pipette of the saliva collection system of FIG. 1 being deposited into the container of the system;

FIG. 3, is a side view of the saliva collection system of FIG. 1; and

FIG. 4, is a perspective view of a transfer pipette of the saliva collection system.

### DETAILED DESCRIPTION OF THE INVENTION

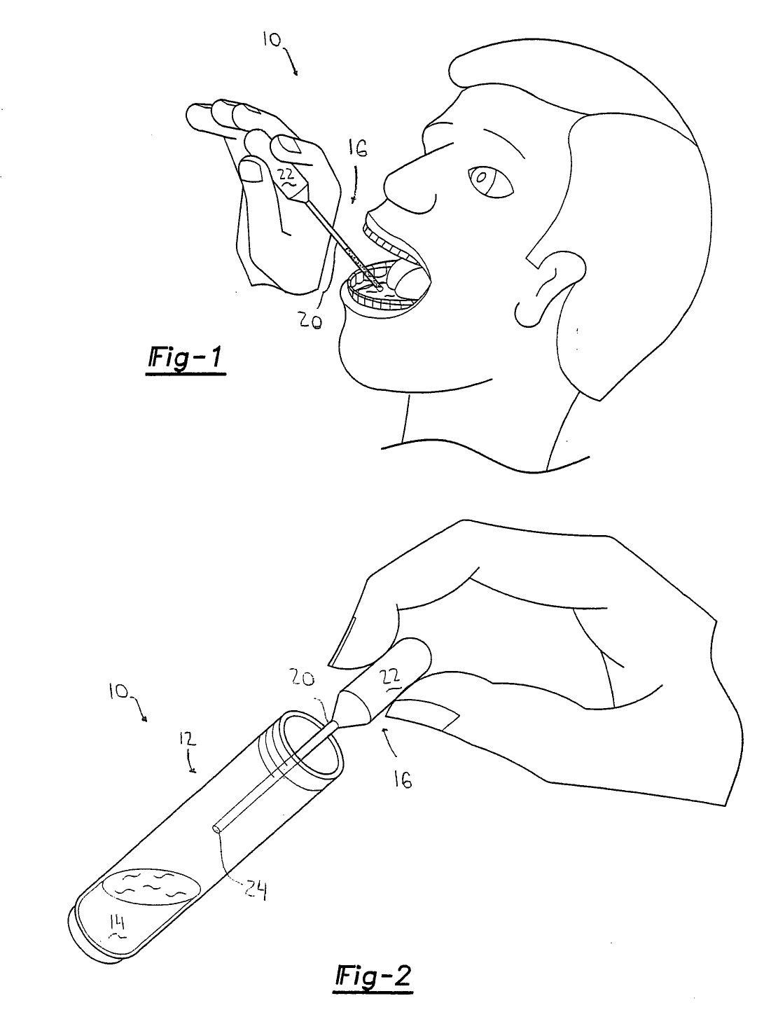

The instant invention provides a kit and method for collecting and preserving a saliva sample for subsequent biologic assay that avoids collection contact with the biohazardous saliva sample. Referring now to FIGS. 1-4, there is shown a saliva collection kit 10 including a container 12, a preservative solution 14 that is retained in the container 12 and a saliva collection device 16. A subsequent laboratory assay performed by conventional techniques specific to the biologic of interest provides results comparable to those obtained from blood or urine samples.

While the description herein of the instant invention is generally directed to hydrophilic compounds, it is appreciated that broad classes of biologics including virus infected epithelial cells, tuberculosis bacilla, carbohydrates, nucleic acids, lipids, fatty acids, sex hormones, cholesterol, insulin, antibodies, peptides, proteins, neurotransmitters and metabolites thereof are detectable in saliva samples. Correlations between saliva and blood plasma concentration of a biologic are readily deduced using the protocols of A. W. Jones Clin. Chem. (1993) Vol. 39(9):1837-1843.

In regard to pathogenic biologics of the instant invention, saliva contains virus or degraded components thereof from virus, illustratively including human immunodeficiency virus (HIV), hepatitis, herpes, influenza, rhinoviruses, adenoviruses, enteroviruses and picornaviruses. Live bacteria or degraded components thereof, illustratively including tuberculosis bacilli, Pneumococcae, Klebsiella bacilli, Streptococci, Staphlococcae, Mycobacteria, Bordetellae, Corynebacteria, Clostridia, Fusobacteria, Escherichae, Spirochetae, Salmonellae, Enterobacteria, Shigellae, and Brucella. The instant invention is particularly well suited by detecting pathogenic biologics infecting buccal and respiratory tract epithelial cells. Such pathogenic biologics are readily detected from saliva collected and preserved according to the present invention using conventional nucleic acid testing or ELISA assays and protocols.

Concentrations of physiological blood plasma biologics are also discerned through saliva analysis. Physiological blood plasma biologics illustratively include carbohydrates; nucleic acids; lipids; fatty acids; melanin; polypeptides; blood groups; prostaglandin; insulin; glucogen; hormones and steroids illustratively including growth hormone releasing factor, endocrine, hypothalamus, pituitary and adrenal gland produced hormones, sex hormones, gastrointestinal hormones, anabolic steroids, clotting factor and steroids active in immune response and reproduction; cholesterol and peptides.

Assay techniques subsequent to sample collection according to the present invention are those conventional to the art and particular to the biologic of interest. Illustrative assay techniques include well plate multiple assay tests, gas-chromatography, mass-spectroscopy, dioxetane luminescence, fluorescence, radiolabelling, antibody binding, polymerase chain reaction amplification and sequencing, ELISA, TSA (NEN Life Sciences) and other methods which are detailed generally in Clinical Diagnosis and Management by Laboratory Methods, 19.sup.th Edition, edited by J. B. Henry (1996) and in chapter 33 thereof in particular.

In assaying for biologics having a molecular weight of less than about 1,500 Daltons from the complex mixture that makes up saliva, a preserved saliva solution is optionally passed through a membrane cut off filter or a chromatography column to a size selected for biologics of interest. Preferably, a fiber mesh membrane filter is used (such as NYLA FLO, Gelman Sciences). A saliva collection kit 10 of the instant invention is capable of preserving for analysis biologics and fragments thereof having a molecular weight of greater than about 100 Daltons.

The instant invention is also operative in preserving for assay biologics including polyclonal antibodies, monoclonal antibodies, major histocompatibility complex (MHC), molecular probes and fragments thereof. Assays using the preserved saliva solution of the instant invention are conducted by methods detailed in chapter 56 of Clinical Diagnosis and Management by Laboratory Methods, 19.sup.th Edition.

Many hydrophilic compounds in general and alcohol in particular, once absorbed from the intestinal tract, and into the bloodstream are evenly mixed into the total body water of the body. For the purpose of the instant invention, a hydrophilic compound is defined as a substance that is found in the body plasma, either in the administered form or as a metabolite thereof. While the description details a method and composition for the preservation of a saliva sample for determination of ethanol content, it is appreciated that the instant invention is operative for the measurement of various other hydrophilic compounds absorbed and excreted by the parenchyma. These other compounds illustratively include: protein; mucin; marijuana, opiates, cocaine, cannibinoids, metabolites thereof; catecholamine and catecholamine derivatives. Fat tissues include tissues or tissue fractions bounded by lipid membranes such as erythrocytes. Hydrophilic compounds enter such a tissue, but are not dissolved into the fat, but rather into the water contained within that tissue. Thus, alcohol for example, is entirely found after several circulation times to be in a volume of approximately 0.60-0.68 liters/kg in a male, and about 0.52-0.54 liter/kg in a female. Once in the body water, alcohol is distributed throughout this volume of water and is subjected to metabolism, excretion, partitioning and excretion limits.

Some parts of blood, especially the water component of blood, are essential for the perfusion of glandular tissues such as the exocrine glands of the alimentary tract, those glands of the mouth and buccal cavity, the pancreas and other organs lower in this path. In particular, the perfusion of the salivary glands of the pharyngeal and buccal cavity, including the parotid glands, the submaxillary glands, and the sublingual glands are of importance to this method. During the process of the blood perfusing these glands, nutrients (amino acids, carbohydrates and fats) and bulk water are taken from the capillary bed(s) of these glands, and are exposed to the individual cells of the gland. Such cells are commonly called the “parenchymal” cells of the gland, e.g., the cells that “secrete” water, protein or other substances (mucin, etc.). It is the “bulk water” fraction, e.g., the water present in the parenchyma, that composes the fluid portion of any secretion from a gland. Finally, a substance dissolved within the “bulk water” of the gland, is often excreted when the gland is called upon to excrete. In the case of any of the salivary glands, water, and some protein material is excreted into the saliva. Thus, excretions of the salivary glands are composed of an isotonic or slightly hypertonic aqueous salt solution, generated from blood plasma. These excretions can also contain various enzymes as are characteristic to the gland, the various enzymes having proteolytic activity to break down or metabolize proteins to peptides and/or amino acids; complex carbohydrate cleavage properties; and to a lesser extent lipid metabolizing properties.

Ethyl alcohol (“alcohol”), when present in the plasma (or blood) from the consumption of ethanol, is a component of the blood that perfuses the salivary glands. It is known that alcohol is extracted into the saliva and that it is concentrated from the plasma during this process, so that, in humans, there is a concentration of 8-15 percent over the concentration present in an equivalent blood sample. Saliva ethanol content has been measured to be about 9 percent higher than in capillary blood, C. Lenter, Geigy Scientific Tables, Vol. 1, Units of Measurement, Body Fluids, Compositions of the Body, Nutrition, Basle: Ciba-Geigy, 1981. In a saliva sample, measurement of blood alcohol level is determined by quantifying the alcohol concentration in a saliva sample.

Studies indicate a high correlation between ethanol concentrations in simultaneously drawn blood, breath and saliva samples. A correlation coefficient of r=0.97 was measured between blood and saliva. A mean saliva-blood concentration difference of 9.4 concentration percent was observed. Statistically, at a 95 percent confidence level saliva alcohol concentration ranges from 88 to 136 concentration percent of the simultaneous blood alcohol level (BAL). A. W. Jones, Clin. Chem. (1993), Vol. 39(9): 1837-1843.

Studies performed by the inventor have determined that the volume of any random “spit” of saliva from the mouth will average approximately 2.0 ml, typically ranging from 1.85 to about 2.35 ml. Such a sample of saliva can be used for the estimation of the concentration of alcohol present in the blood that perfused the salivary glands producing the saliva sample.

The sample of saliva when caught and preserved in a suitable solution is subsequently used to estimate a blood concentration of alcohol in the person from whom it is taken.

Referring now to FIGS. 1-4, there is shown a saliva collection kit 10 including a container 12, a preservative solution 14 that is retained in the container 12 and a saliva collection device 16.

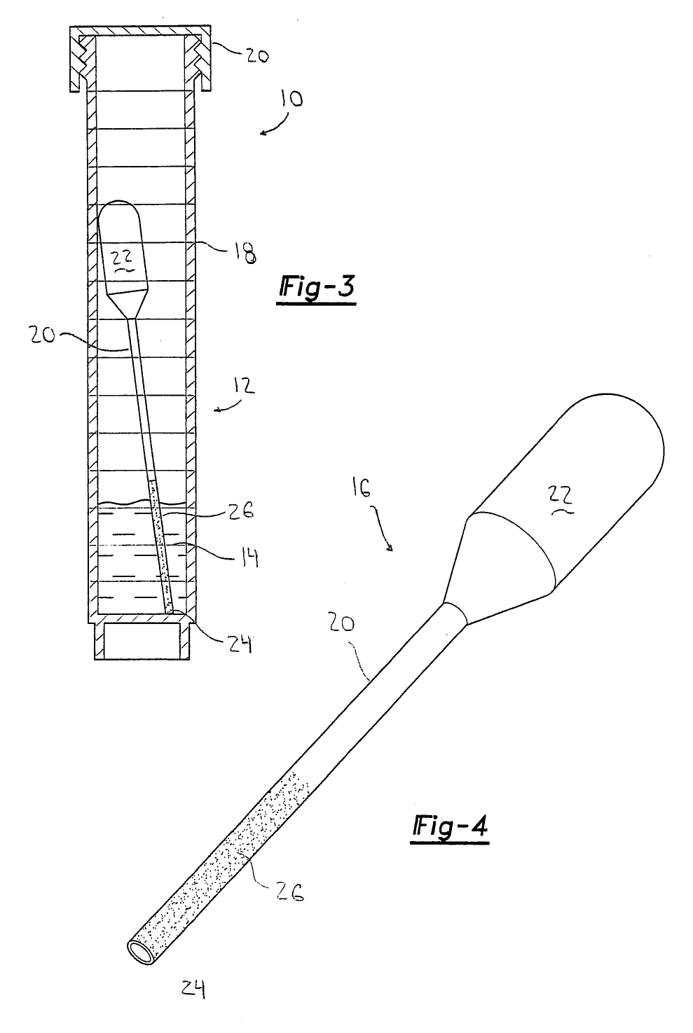

Referring now to FIGS. 2 and 3, the container 12 of the present invention is a resealable tube. The resealable tube may be a 15 ml polyethylene transfer vial 18 having a screw cap 20. Such vials 18 are available from Evergreen Scientific of Los Angeles, Calif.

Still referring to FIGS. 2 and 3, the preservative solution 14 into which the saliva is placed includes an agent for lessening the degradation of the saliva by the inhibition of enzymatic metabolism of the alcohol or test substance present in the sample by bacteria, fungi, white blood cells, macrophages, or other organisms that can reside in the environment of the buccal or respiratory cavities of the sample donor. The solution may contain an ionic solute present at a concentration in the range of osmalities associated with normal physiological body fluids. The body fluids including body plasma, urine and saliva. Approximately, 3.0 ml (+/−0.5 ml) of the preservative solution 14 is dispensed into each container 12. The preservative solution 14 may be a buffered preservative solution prepared according to the following instructions. To 900 ml of water, add 8.5 gm sodium chloride (NaCl) with NaHPO₄ and NaH₂PO₄ in a concentration to provide 50 mM phosphate solution. To this solution is added 0.5-2.0 gm sodium benzoate (NaC₇H₅O₂). The entire solution is then dissolved QS to 1000 ml and pH with 10 N NaOH to a pH of 6.2.

The preservative solution 14 serves to arrest the action of enzymes that degrade substances such as drugs or alcohol within living cells contained in the sample or in the solution of the specimen cup. In embodiments of the instant invention operative in determining alcohol concentration, the inhibition of alcohol dehydrogenase is of particular concern. Representative enzymatic inhibiting agents of the instant invention include: aminoglycosides, cephelosporins, tetracyclines, sulfa-drugs, penicillin and similar antibiotics.

It is appreciated that the optimal preservative solution 14 concentration is dictated by the efficacy of the specific compound in disrupting enzymatic activity. The agents of the instant invention also may have secondary biocidal effects on organisms present in the specimen cup. Preferably, the enzymatic inhibiter functions to interfere with glycolysis pathway reactions.

Optionally, a fungicide (or mycocide) is added to the preservative solution 14. Preferably, the fungicide (or mycocide) is present in a concentration from about 0.01 to 10 mole percent, relative to the specimen solution water. More preferably, the fungicide (or mycocide) is present in a concentration from about 0.05 to 1 mole percent, relative to the specimen solution water. Fungicides or mycocides operative in the instant invention illustratively include: polymyxins, polynoxylins, nystatin, hedaquinium chlorides, tetrachloroisophtalonitrile and ketoconazole.

Optionally, a bactericide is added to the preservative solution 14. Preferably, the bactericide is present in a concentration from about 0.01 to 10 mole percent, relative to the specimen solution water. More preferably, the bactericide is present in a concentration from about 0.05 mole percent, relative to the specimen solution water. Bactericides operative in the instant invention illustratively includes: aninoglycosides, cephelosporins, tetracyclenes, sulfa-drugs, pencillins and similar antibiotics.

The order by which the above reagents are prepared or mixed is not essential and has no bearing on the ultimate utility of the solution in the instant invention.

Other preservatives may also be used. Such other preservatives include those identified and described in U.S. Pat. Nos. 5,968,746 and 6,291,178, the disclosures of which are incorporated herein in their entireties by reference.

Still referring to FIGS. 1-4, the saliva collection device 16 may be a 1 ml polyethylene transfer pipette 20 having a compression end 22 and an intake end 24. Suitable transfer pipettes 20 are available from Evergreen Scientific of Los Angeles, Calif.

As best shown in FIG. 4, the intake end 24 of the transfer pipette 20 is coated with a salivation catalyst 26. The salivation catalyst is a coating of a 20 percent solution of lemon extract (1 ml diluted to 5 ml final volume) that is applied by spray to the intake end 24 of the transfer pipette. Once applied the extract is dried using a heat lamp (not shown) or the like. Alternatively, the catalyst 26 may be a spearmint, orange or peppermint flavor. Suitable flavorings are available under the name Frontier Natural Flavors sold by Frontier Co-Op of Norway, Iowa. Alternatively, suitable flavorings are available from Boyajian, Inc. of Newton, Mass. It will also be appreciated that natural or artificial flavors may also be used as the catalyst 26.

In those instances where the salivation collection device 16 is unsterilized, certain steps of manufacture may be necessary in order to prepare the collection device 16 for use. Therefore, in a first step the unsterilized collection device 16 is coated with the salivation catalyst 26. As stated above, this first step is accomplished by spraying the collection device 16 with a flavored solution and then drying the collection device 16 under a heat lamp. Thereafter, the collection device 16 is sterilized through the use of an autoclave, irradiation, exposure to ethylene oxide or the like. Once sterilized, the collection device 16 is placed in sterile packaging. Suitable sterile packaging includes the self-sealing sterilization pouch sold by CROSSTEX® International. The collection device may then be packaged with the container 12, having the solution 14 therein, for later administration.

In operation, a user of the preferred embodiment will place the intake end 24 of a transfer pipette 20 (or other the collection device 16) in his or her mouth (FIG. 1), preferably beneath the tongue. Once inserted, the sour or sweet taste of the salivation catalyst 26 quickly promotes a salivation response from the user. Allowing the pipette 20 to remain in place for between 60 and 90 seconds should permit an amount of saliva to pool in the users mouth. At this point, it is important that the user not swallow the saliva.

After the user has begun to salivate, he or she depresses and release the compression end 22 of the transfer pipette 20 to draw a saliva sample (not shown) into the transfer pipette 20. This process may be repeated if necessary.

Once a sample of saliva has been drawn into the transfer pipette 20, the entire transfer pipette 20 is placed into the container 12 (FIG. 2). The container 12 is then sealed (FIG. 3) and shipped/transferred to a testing facility where a sample may be extracted from the saliva/preservative solution for diagnostic testing on a mass spectrometer or the like. Shipping/transfer packaging for the container 12, with the transfer pipette 20 may include labels that seal and/or allow for tracking and identification of the transferred container 12/test.

Having thus described my invention, various other embodiments and improvements will become apparent to those having skill in the art which do not depart from the scope of the present invention.

I claim:

1. A kit for the collection and preservation of a saliva sample for subsequent assay of a biologic therein comprising:
a container;
a saliva collection device, said saliva collection device including a salivation catalyst; and
a preservative solution retained within said container.

2. The kit of claim 1, wherein the container comprises a resealable tube.

3. The kit of claim 2, wherein the resealable tube comprises a polyethylene transfer vial.

4. The kit of claim 1, wherein the saliva collection device comprises a transfer pipette having a compression end and an intake end.

5. The kit of claim 1, wherein the salivation catalyst comprises a food flavoring.

6. The kit of claim 5, wherein the food flavoring is selected from a group consisting of lemon, peppermint, spearmint and orange flavorings.

7. The kit of claim 1, wherein the preservative solution comprises:
sodium chloride, NaHPO₄ and NaH₂PO₄ in an aqueous concentration to provide a 50 mM phosphate solution; and
0.5-2.0 g sodium benzoate.

8. The kit of claim 7, wherein the preservative solution comprises a pH of 6.2.

9. The kit of claim 1, wherein the preservative solution comprises a fungicide.

10. The kit of claim 1, wherein the preservative solution comprises a bactericide.

11. A method of assaying for a biological comprising the steps of:
providing a container;
providing a preservative solution within the container;
providing a saliva collection device, the saliva collection device including a salivation catalyst;
collecting at a first location a saliva sample directly from a mouth of a user using the saliva collection device;
depositing the saliva collection device having the saliva sample into the container; and sealing the container having the saliva collection device against spillage and tampering.

12. The method of claim 11, further comprising the step of shipping the container having the saliva collection device to a second location.

13. The method of claim 12, further comprising the step of assaying the saliva sample and preservative solution subsequently for a biologic at a second location.

14. The method of claim 11, further comprising the step of assaying the saliva sample and preservative solution subsequently for a biologic at a second location.

15. The kit of claim 11, wherein the container comprises a resealable tube.

16. The kit of claim 15, wherein the resealable tube comprises a polyethylene transfer vial.

17. The kit of claim 11, wherein the saliva collection device comprises a transfer pipette having a compression end and an intake end.

18. The kit of claim 11, wherein the salivation catalyst comprises a food flavoring.

19. The kit of claim 18, wherein the food flavoring is selected from a group consisting of lemon, peppermint, spearmint and orange flavorings.

20. The kit of claim 11, wherein the preservative solution comprises:
sodium chloride, NaHPO₄ and NaH₂PO₄ in an aqueous concentration to provide a 50 mM phosphate solution; and
0.5-2.0 g sodium benzoate.

21. The kit of claim 11, wherein the preservative solution comprises a fungicide.

22. The kit of claim 11, wherein the preservative solution comprises a bactericide.
